(19) United States
(12) Patent Application Publication
(10) Pub. No.: US 2025/0025608 A1
(43) Pub. Date: Jan. 23, 2025

(54) METAL WITH MICROPOROUS SURFACE STRUCTURE, PREPARATION METHOD THEREFOR, AND APPLICATION THEREOF

(71) Applicant: YANSHAN UNIVERSITY, Qinhuangdao (CN)

(72) Inventors: Hongwang ZHANG, Qinhuangdao (CN); Xin WANG, Qinhuangdao (CN); Zhaoji WANG, Qinhuangdao (CN)

(73) Assignee: YANSHAN UNIVERSITY, Qinhuangdao (CN)

(21) Appl. No.: 18/282,549

(22) PCT Filed: Apr. 4, 2023
(86) PCT No.: PCT/CN2023/086163

§ 371 Date: Sep. 18, 2023

(30) Foreign Application Priority Data
Apr. 7, 2022 (CN) 202210360096.7

(51) Int. Cl. A61L 31/14; A61L 31/02; C23F 4/00

(52) U.S. Cl. CPC A61L 31/146; A61L 31/022; A61L 2400/18; C23F 4/00

(57) ABSTRACT

The present invention belongs to the technical field of mechanical manufacturing and medical device surface processing, and specifically discloses a metal with a microporous surface structure, a preparation method therefor, and an application thereof. According to the present invention, a metal is sequentially sanded and polished, and then subjected to ultrasonic cavitation, wherein conditions for the ultrasonic cavitation are as follows: the ultrasonic cavitation is performed for 1-240 min, an amplitude of ultrasonic equipment is 1-100 μm, an output power of the ultrasonic equipment is 5-200 W, and an ultrasonic frequency is 15000-55000 Hz; and the metal with a microporous surface structure is obtained. The method disclosed by the present invention has the advantages of low cost, simple operation, and no pollution to the surface, can solve the preparation of micropores on small and micro surfaces, and has potential wide application prospects in medical biology and other related fields.

### TECHNICAL FIELD

The present invention relates to the technical field of mechanical manufacturing and medical device surface processing, and in particular, to a metal with a microporous surface structure, a preparation method therefor, and an application thereof.

### BACKGROUND

Many metal materials such as titanium, a titanium alloy, and stainless steel are widely used as medical material biological implants, such as dental nails, joints, and various permanent fixtures due to their excellent mechanical properties, such as high strength, high bending fatigue strength, elastic modulus similar to human skeleton, good toughness, and excellent biocompatibility. Generally, after the medical material is implanted into a living body, a biological tissue can directly act with a surface of the material (D. A. Puleo, A. Nanci, Biomaterials, 1999, 20:2311). For example, long-term observation of the clinical use state of titanium implants shows that the titanium alloy can cause corresponding biological reaction after being implanted into a living body, so that the wrapping is formed around the implant, and an interface between a bone tissue of the living body and the material can be clearly observed under a microscope. Research shows that a surface of an implant is provided with a microporous structure with a proper size, so that the adhesion and proliferation capacity of living cells can be improved, the adhesion of the cells and the growth of a microvascular structure are facilitated, and a compact interface structure is formed. The preparation of micropores on the surface of metal materials is an important problem.

The preparation technology for the microporous surface mainly comprises a mechanical method and a chemical method. The most commonly used mechanical methods are surface shot blasting and laser surface texturing, wherein the surface shot blasting uses high-hardness shot with a diameter of dozens or hundreds of micrometers, and the shot impacts a metal surface at a high speed by a sandblasting machine to form a rough surface. The laser surface texturing uses a high-energy-density laser beam to scan a surface of a workpiece to process a specific structure with a regular and uniform appearance. For example, Chinese Patent Application No. CN200810017825.9 discloses a micropore processing method for a rough surface of a bone restoration. This method firstly uses a laser engraving machine with an output power of 10-25 W to etch a plurality of micropores that are uniformly and continuously distributed on a surface of a bone restoration by a conventional laser etching method, wherein the micropore has a cross-sectional area of 1950-200000 μm² and a depth of 5-500 μm, then the etched bone restoration is put into an acid solution prepared by HF, HNO₃ and H₂O or HNO₃, HF, H₂O₂, and H₂O to chemically wash a rough microporous surface, and then the rough microporous surface is cleaned by water, so that a uniform and continuous rough microporous structure is formed on the surface of the implant. The chemical methods mainly comprise micro-arc anodic oxidation and alkali heat treatment. The micro-arc anodic oxidation is to form a microporous structure on a surface of a to-be-treated metal by using an electrochemical discharge principle, and the alkali heat treatment is to immerse a to-be-treated implant in a high-temperature alkaline solution to cause the surface of the implant to generate alkali corrosion so as to generate the microporous structure. Chinese Patent Application No. CN200910112256.0 discloses a surface treatment method for improving biological performance of metal titanium. This method comprises the steps of pretreating a surface of a titanium metal substrate, performing alkali treatment on the titanium metal substrate in a NaOH solution at a temperature of 100-150° C. for 2-6 h to obtain a nano-micron ordered sodium titanate film layer on the surface of the titanium metal substrate, and calcining the titanium metal substrate with the nano-micron ordered sodium titanate film layer on the surface at a temperature of 450° C. for 2 h to obtain an anatase TiO₂ film layer with a specific structure.

Although the foregoing methods can prepare a microporous structure on a metal surface, these methods have many defects, for example, a shot blasting method causes pollution to the surface to a certain degree and has a high processing difficulty on small and micro complex surfaces; the laser surface texturing is expensive in equipment and high in cost, and acid pickling treatment is required after laser etching; and the alkali heat treatment process is complicated and time-consuming.

Therefore, how to provide a metal with a microporous surface structure, a preparation method therefor, and an application thereof that can simplify the metal surface processing difficulty, reduce pollution, reduce cost, and shorten process time is a difficult problem to be solved urgently in the art.

### SUMMARY

In view of this, the present invention provides a metal with a microporous surface structure, and a preparation method therefor, and an application thereof. The present invention simplifies the process, reduces the pollution and cost of the process, and shortens the processing time of the metal.

In order to achieve the above objective, the present invention uses the following technical solutions.

A preparation method for a metal with a microporous surface structure comprises the following steps:
1) sequentially sanding and polishing the metal;
2) performing ultrasonic cavitation on the metal polished in the step 1) to obtain the metal with the microporous surface structure;
wherein micropores of the metal with the microporous surface structure have an aperture of 8-50 μm.

Preferably, the metal comprises titanium, a titanium alloy, or stainless steel.

Preferably, the sanding in the step 1) refers to first sanding with 1000 #SiC waterproof abrasive paper for 8-15 min; then use 2000 #SIC abrasive paper to polish for 8-15 min; finally, use 3000 #SiC waterproof abrasive paper for 8-15 min.

Preferably, the polishing is performed for 20-60 min.

Preferably, the metal is located right below an ultrasonic cavitation tool head at a distance of 0.5-50 mm during the ultrasonic cavitation.

Preferably, the conditions for the ultrasonic cavitation are as follows: the ultrasonic cavitation is performed for 1-240 min, an amplitude of the ultrasonic equipment is 1-100 μm, an output power of the ultrasonic equipment is 5-200 W, and an ultrasonic frequency is 15000-55000 Hz.

Another objective of the present invention is to provide a metal with a microporous surface structure obtained through the preparation method for a metal with a microporous surface structure.

Still another objective of the present invention is to provide an application of the metal with a microporous surface structure as a medical material biological implant.

It can be known from the technical solutions that, compared with the prior art, the present invention has the following beneficial effects:

The present invention uses the high-frequency current generated by an ultrasonic generator to generate high-frequency longitudinal vibration with a frequency of up to 15000-55000 Hz through an ultrasonic transducer and an ultrasonic amplitude transformer so as to induce the violent cavitation effect in the liquid medium. The 1000 times/(s·cm²) high-pressure shock waves and a micro-jet with a speed as high as 100 m/s generated at the moment of collapsing the vacuoles impact a surface of the material, so that cavitation erosion occurs on the surface of the material, and the microporous surface structure is formed. The method disclosed by the present invention has the advantages of low cost, simple operation, and no pollution to the surface, can solve the preparation of micropores on small and micro surfaces, and has potential wide application prospects in medical biology and other related fields.

The equipment used in the present invention is simple, low in cost, and convenient for large-scale popularization and application; and the conditions have no strict requirements, and can be performed at room temperature.

The metal with the microporous structure prepared by the present invention has a pore size of several to dozens of micrometers, can significantly improve the adhesion and proliferation capacity of living cells, is beneficial to the adhesion of the cells and the growth of the microvascular structure, and thus forms a compact interface structure.

### BRIEF DESCRIPTION OF THE DRAWINGS

In order to more clearly illustrate the technical solution in the examples of the present invention or in the prior art, the drawings required to be used in the description of the examples or the prior art are briefly introduced below. It is obvious that the drawings in the description below are merely examples of the present invention, and those of ordinary skill in the art can obtain other drawings according to the drawings provided without creative efforts.

FIG. 1 is an optical microscope image of a titanium metal surface after mechanical polishing;

FIG. 2 is a scanning electron microscopy image of a titanium metal surface after ultrasonic cavitation;

FIG. 3 is a schematic view of ultrasonic cavitation equipment; wherein 1 represents an ultrasonic generator, 2 represents a lifting platform, 3 represents an ultrasonic transducer, 4 represents an ultrasonic amplitude transformer, 5 represents an ultrasonic tool head, 6 represents a metal sample, and 7 represents cavitation liquid;

FIG. 4 is a schematic diagram of an ultrasonic cavitation principle; and

FIG. 5 is a graph of experimental results of cell growth of a metal pure titanium with a microporous surface structure prepared in Example 1 and medical titanium, wherein the left panel corresponds to the medical titanium, and the right panel corresponds to the metal pure titanium with the microporous surface structure prepared in Example 1.

### DETAILED DESCRIPTION OF THE EMBODIMENTS

The present invention provides a preparation method for a metal with a microporous surface structure comprising the following steps:
1) sequentially sanding and polishing the metal;
2) performing ultrasonic cavitation on the metal polished in the step 1) to obtain the metal with the microporous surface structure;
wherein micropores of the metal with the microporous surface structure have an aperture of 8-50 μm.

In the present invention, a cleaning step is also involved after polishing, and the cleaning step is to clean the metal by sequentially using acetone, ethanol, and ultrasonic cleaning.

In the present invention, after the ultrasonic cavitation is completed, a cleaning step is also included, which aims to clean the residue introduced by the ultrasonic cavitation in the step 2), and the cleaning step is to clean the metal by sequentially using acetone, ethanol, and ultrasonic cleaning.

In the present invention, the cavitation liquid is preferably deionized water.

In the present invention, the metal comprises titanium, a titanium alloy, or stainless steel.

In the present invention, the sanding in the step 1) refers to first sanding with 1000 #SiC waterproof abrasive paper for 8-15 min; then use 2000 #SIC abrasive paper to polish for 8-15 min; finally, use 3000 #SiC waterproof abrasive paper for 8-15 min.

In the present invention, the polishing is performed for 20-60 min, preferably 30-50 min, and further preferably 40 min.

In the present invention, during the ultrasonic cavitation, the metal is located right below an ultrasonic cavitation tool head at a distance of 0.5-50 mm, preferably 10-30 mm, and further preferably 20 mm.

In the present invention, the conditions for the ultrasonic cavitation are as follows: the ultrasonic cavitation is performed for 1-240 min, preferably 1-5 min, and further preferably 3 min; an amplitude of the ultrasonic equipment is 1-100 μm, preferably 2-50 μm, and further preferably 5 μm; an output power of the ultrasonic equipment is 5-200 W, preferably 8-100 W, and further preferably 10 W; and an ultrasonic frequency is 15000-55000 Hz, preferably 20000-50000 Hz, and further preferably 40000 Hz.

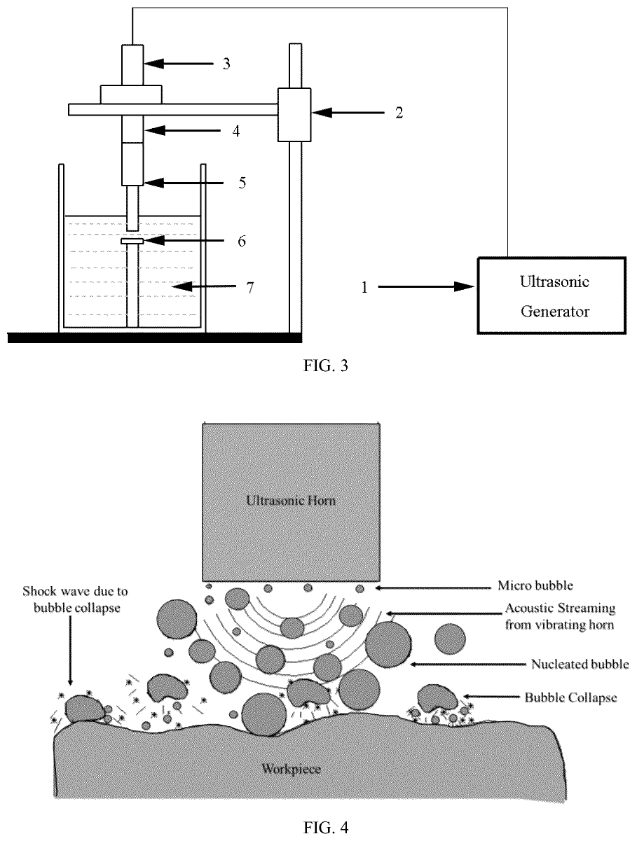

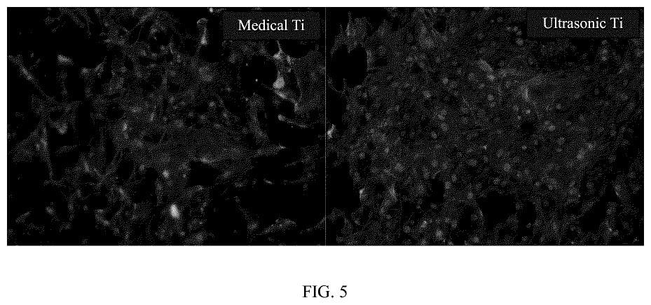

The schematic diagram of the ultrasonic cavitation equipment of the present invention is shown in FIG. 3. The ultrasonic cavitation equipment comprises an ultrasonic generator 1, a lifting platform 2, an ultrasonic transducer 3, an ultrasonic amplitude transformer 4, an ultrasonic tool head 5, a metal sample 6, and a cavitation liquid 7. The schematic diagram of the ultrasonic cavitation principle is shown in FIG. 4.

The present invention further provides a metal with a microporous surface structure obtained through the preparation method for a metal with a microporous surface structure.

The present invention further provides an application of the metal with a microporous surface structure obtained through the preparation method for a metal with a microporous surface structure as a medical material biological implant.

The technical solutions in the examples of the present invention will be clearly and completely described below. Apparently, the described examples are merely a part, rather than all of the examples of the present invention. Based on the examples of the present invention, all other examples obtained by those of ordinary skill in the art without creative effort fall within the protection scope of the present invention.

### Example 1

The pure titanium was used as a treatment metal, and this metal was sanded for 10 min by using 1000 #SiC waterproof abrasive paper, then sanded for 10 min by using 2000 #SiC waterproof abrasive paper, and then sanded for 10 min by using 3000 #SiC waterproof abrasive paper. The sanded metal was polished for 30 min by using a polishing machine, the polished metal material was cleaned by sequentially using acetone, ethanol and ultrasonic cleaning, surface debris was removed, and the metal material was dried for later use, wherein an optical microscope image of the polished pure titanium surface is shown as FIG. 1, and it can be seen from FIG. 1 that the polished titanium metal surface is smooth and flat.

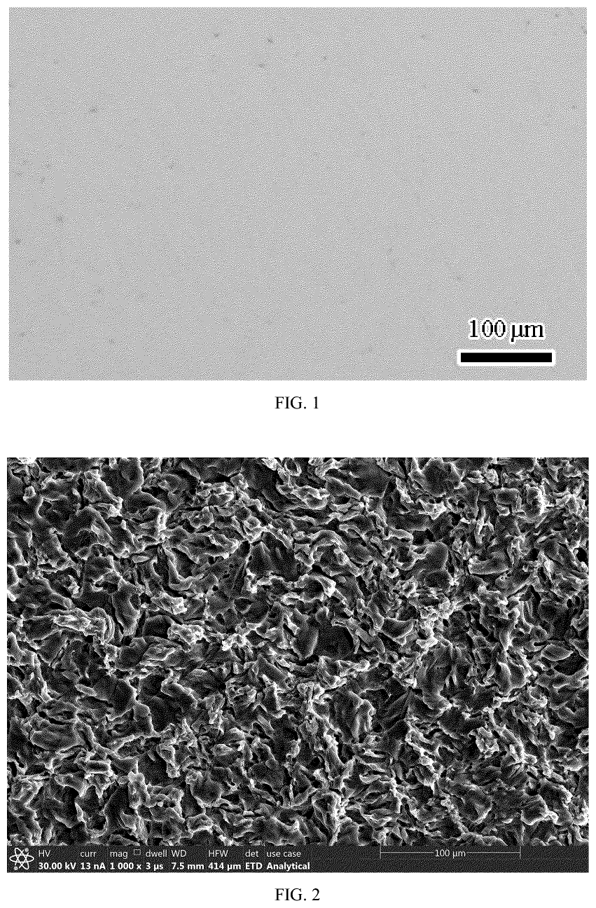

The treated metal material was placed right below an ultrasonic cavitation tool head at a distance of 20 mm for ultrasonic cavitation treatment, wherein the conditions for the ultrasonic cavitation are as follows: the ultrasonic cavitation is performed for 3 min, an amplitude of the ultrasonic equipment is 5 μm, an output power of the ultrasonic equipment is 10 W, and an ultrasonic frequency is 40 KHz. The scanning electron microscopy image of the titanium metal surface after ultrasonic cavitation is shown in FIG. 2. It can be found by comparing FIG. 1 and FIG. 2 that the titanium metal surface is significantly changed, and a large number of holes with a diameter of 8-50 μm can be observed on the titanium metal surface.

After the ultrasonic cavitation was completed, the metal was cleaned by sequentially using acetone, ethanol, and ultrasonic cleaning to remove residues introduced by the ultrasonic cavitation to obtain the metal with the microporous surface structure.

### Example 2

The TC4 (titanium alloy) was used as a treatment metal, and this metal was sanded for 8 min by using 1000 #SiC waterproof abrasive paper, then sanded for 15 min by using 2000 #SiC waterproof abrasive paper, and then sanded for 10 min by using 3000 #SiC waterproof abrasive paper. The sanded metal was polished for 30 min by using a polishing machine, the polished metal material was cleaned by sequentially using acetone, ethanol and ultrasonic cleaning, surface debris was removed, and the metal material was dried for later use.

The treated metal material was placed right below an ultrasonic cavitation tool head at a distance of 50 mm for ultrasonic cavitation treatment, wherein the conditions for the ultrasonic cavitation are as follows: the ultrasonic cavitation is performed for 240 min, an amplitude of the ultrasonic equipment is 100 μm, an output power of the ultrasonic equipment is 200 W, and an ultrasonic frequency is 15 KHz.

After the ultrasonic cavitation was completed, the metal was cleaned by sequentially using acetone, ethanol, and ultrasonic cleaning to remove residues introduced by the ultrasonic cavitation to obtain the metal with the microporous surface structure.

### Example 3

The 304 stainless steel was used as a treatment metal, and this metal was sanded for 15 min by using 1000 #SiC waterproof abrasive paper, then sanded for 8 min by using 2000 #SiC waterproof abrasive paper, and then sanded for 8 min by using 3000 #SiC waterproof abrasive paper.

The sanded metal was polished for 20 min by using a polishing machine, the polished metal material was cleaned by sequentially using acetone, ethanol and ultrasonic cleaning, surface debris was removed, and the metal material was dried for later use.

The treated metal material was placed right below an ultrasonic cavitation tool head at a distance of 0.5 mm for ultrasonic cavitation treatment, wherein the conditions for the ultrasonic cavitation are as follows: the ultrasonic cavitation is performed for 1 min, an amplitude of the ultrasonic equipment is 1 μm, an output power of the ultrasonic equipment is 5 W, and an ultrasonic frequency is 55 KHz.

After the ultrasonic cavitation was completed, the metal was cleaned by sequentially using acetone, ethanol, and ultrasonic cleaning to remove residues introduced by the ultrasonic cavitation to obtain the metal with the microporous surface structure.

### Experiment Example 1

The metal pure titanium with the microporous structure prepared in Example 1 was subjected to the cell growth experiment and compared with the medical Ti of the same material used in the actual implant. The experiment result is shown in FIG. 5, wherein the right panel corresponds to Example 1 of the present invention. The blue dots in the figure are the stained nuclei. It can be found that the cell adhesion growth on the Ti surface after ultrasonic cavitation is more uniform and dense.

The examples in the specification are all described in a progressive manner, and each example focuses on differences from other examples, and portions that are the same and similar between the examples may be referred to each other.

The above description of the disclosed examples enables those skilled in the art to implement or use the present invention. Various modifications to these examples will be readily apparent to those skilled in the art, and the generic principles defined herein may be applied to other examples without departing from the spirit or scope of the present invention. Thus, the present invention is not intended to be limited to these examples shown herein but is to be accorded the broadest scope consistent with the principles and novel features disclosed herein.

1. A preparation method for a metal with a microporous surface structure, comprising the following steps:
1) sequentially sanding and polishing the metal;
2) performing ultrasonic cavitation on the metal polished in the step 1) to obtain the metal with the microporous surface structure;
wherein micropores of the metal with the microporous surface structure have an aperture of 8-50 μm;
conditions for the ultrasonic cavitation are as follows: the ultrasonic cavitation is performed for 1-240 min, an amplitude of ultrasonic equipment is 1-100 μm, an output power of the ultrasonic equipment is 5-200 W, and an ultrasonic frequency is 15000-55000 Hz.

2. The preparation method for a metal with a microporous surface structure according to claim 1, wherein the metal comprises titanium, a titanium alloy, or stainless steel.

3. The preparation method for a metal with a microporous surface structure according to claim 2, wherein the sanding in the step 1) refers to first sanding with 1000 #SiC waterproof abrasive paper for 8-15 min; then use 2000 #SIC abrasive paper to polish for 8-15 min; finally, use 3000 #SiC waterproof abrasive paper for 8-15 min.

4. The preparation method for a metal with a microporous surface structure according to claim 3, wherein the polishing is performed for 20-60 min.

5. The preparation method for a metal with a microporous surface structure according to any one of claims 1 to 4, wherein the metal is located right below an ultrasonic cavitation tool head at a distance of 0.5-50 mm during the ultrasonic cavitation.

6. A metal with a microporous surface structure obtained through the preparation method for a metal with a microporous surface structure according to any one of claims 1 to 5.

7. An application of the metal with a microporous surface structure according to claim 6 as a medical material biological implant.
